(19) United States
(12) Patent Application Publication
(10) Pub. No.: US 2026/0325067 A1
(43) Pub. Date: Oct. 22, 2026

(54) DEVICE FOR DETECTING AN ANOMALY IN THE WEARING OF A RESPIRATORY MASK

(71) Applicants: OSO-AI, Brest (FR); SORBONNE UNIVERSITE, Paris (FR); INSTITUT NATIONAL DE LA SANTE ET DE LA RECHERCHE MEDICALE, Paris (FR); ASSISTANCE PUBLIQUE HOPITAUX DE PARIS, Paris (FR)

(72) Inventors: Thomas Similowski, Paris (FR); Capucine Morelot-Panzini, Paris (FR); Philippe Roguedas, Brest (FR)

(73) Assignees: OSO-AI, Brest (FR); SORBONNE UNIVERSITE, Paris (FR); INSTITUT NATIONAL DE LA SANTE ET DE LA RECHERCHE MEDICALE, Paris (FR); ASSISTANCE PUBLIQUE HOPITAUX DE PARIS, Paris (FR)

(21) Appl. No.: 19/114,706

(22) PCT Filed: Sep. 22, 2023
(86) PCT No.: PCT/EP2023/076216

§ 371 Date: Mar. 24, 2025

(30) Foreign Application Priority Data
Sep. 22, 2022 (FR) FR2209633

(51) Int. Cl. A61B 5/00; A61B 7/00; A61B 7/04; A61M 16/06

(52) U.S. Cl. CPC A61B 5/4833; A61B 5/7267; A61B 7/003; A61B 7/04; A61M 16/06; A61M 2210/0618; A61M 2210/0625

(57) ABSTRACT

Method for detecting an anomaly in the wearing of a respiratory mask applied against the face of a user, comprising:
a) detecting, by a microphone, a sound produced, at different times, by the user exhaling or inhaling through the mask;
b) processing of the detected sound by a processing unit, at each instant, so as to extract at least one characteristic of the sound;
c) according to each characteristic extracted in step b), detecting, by the processing unit, the anomaly in the wearing of the mask. FIG. 1A.

### TECHNICAL FIELD

The technical field of the invention is the monitoring of users wearing a respiratory mask.

### BACKGROUND ART

Respiratory masks are commonly used to treat respiratory pathologies such as chronic obstructive pulmonary disease, obesity-hypoventilation syndrome, restrictive lung disease and sleep apnea.

One possible function of such masks is to connect the patient wearing it to a respiratory assistance machine delivering excess pressure to the respiratory system, covering the nose or both the nose and mouth. For example, this enables non-invasive ventilation treatment, which provides mechanical assistance for breathing by supplying the mask with pressurized air a cyclic fashion. This reduces the workload on respiratory muscles and improves gas exchange. In another type of treatment, the air is pressurized in a non-cyclical way, in a modality, known as “continuous positive airway pressure”, which can be used to treat sleep apnea.

The effectiveness of the treatment depends on the mask being correctly applied to the face, particularly during periods when the user's vigilance is insufficient, such as during sleep. Depending on the treatment, the mask used may be a face mask, covering the mouth and nose, or a nasal mask, covering the nose only.

Documents WO2021243293, WO2010091462, WO2020118311 and WO2021245637 describe breathing masks for treating sleep apnea. In WO2021243293, the detection of a sound is mentioned, in a spectral band below 1000 Hz, for leak detection purposes,.

It is desirable to have a device that effectively, discreetly, and easily monitors whether a breathing mask is being used correctly. The invention addresses this need.

### SUMMARY OF THE INVENTION

A first object of the invention is a method for detecting an anomaly in the wearing of a respiratory mask applied against the face of a user, comprising:
a) detecting, by a microphone, a sound produced, at different times, by the user exhaling or inhaling through the mask;
b) processing of the detected sound by a processing unit, at each instant, so as to extract at least one characteristic;
c) as a function of each characteristic extracted in step b), detecting, by the processing unit, the occurrence of an anomaly in the wearing of the mask.

Step c) may comprise:
c1) taking into account at least one previously stored criterion is;
c2) comparing each characteristic extracted in step b) with the criterion or one of the criteria taken into account in sub-step c1).

The processing may include:
selecting at least one sound frequency band;
determining a characteristic in the selected frequency band.

One possibility:
the characteristic is a spectral power;
the criterion is a spectral power threshold, so that the anomaly is detected when the spectral power in the selected frequency band exceeds the spectral power threshold.

One possibility:
the characteristic is a spectral power variation;
the criterion is a spectral power variation threshold, so that the anomaly is detected when the spectral power variation, in the selected frequency band, exceeds the spectral power variation threshold.

One possibility:
the criterion is a threshold;
sub-step c1) comprises:
   taking into account instants at which the characteristic crosses the threshold;
   estimating a threshold crossing time period during which the characteristic remains above or below the threshold;
in substep c2), the anomaly is detected when the threshold crossing time period is within a predetermined range.

One possibility:
the mask is a face mask, configured to cover the user's mouth and nose;
the frequency band is greater than 100 Hz.

According to one possibility
the mask is a nasal mask, configured to cover the user's nose without covering the mouth;
the frequency band is between 300 Hz and 13000 Hz.

According to one possibility, the frequency band extends from 1000 Hz, or from 1100 Hz, up to 10000 Hz or 13000 Hz.

One possibility:
the processing unit implements a supervised learning artificial intelligence algorithm;
the supervised learning artificial intelligence algorithm is parameterized by a learning phase that takes into account sounds detected in the absence and presence of an anomaly.

A second object is a device for monitoring the wearing of a breathing mask by a user, the device comprising:
a microphone, configured to record sounds of the user's breathing through the mask;
a processing unit, programmed to receive the sounds recorded by the microphone, and to implement steps b) and c) of the method according to the first object of the invention.

The invention will be better understood on reading the examples of embodiments presented, in the following description, in connection with the figures listed below.

### BRIEF DESCRIPTION OF THE DRAWINGS

The invention will be better understood on reading the examples of embodiments presented, in the following description, in connection with the figures listed below.

FIG. 1A shows an example of a device according to the invention.

FIG. 1B illustrates the use of a face mask, and possible air outlets.

FIG. 2 shows a frequency decomposition of sounds produced by a face mask worn by a user.

FIG. 3A shows a nasal mask.

FIG. 3B shows a nasal mask is worn.

FIG. 4 shows a frequency decomposition of sounds produced by a nasal mask worn by a user.

FIG. 5 shows a schematic diagram of the steps involved in implementing a device according to the invention.

### DETAILED DESCRIPTION OF PARTICULAR EMBODIMENTS

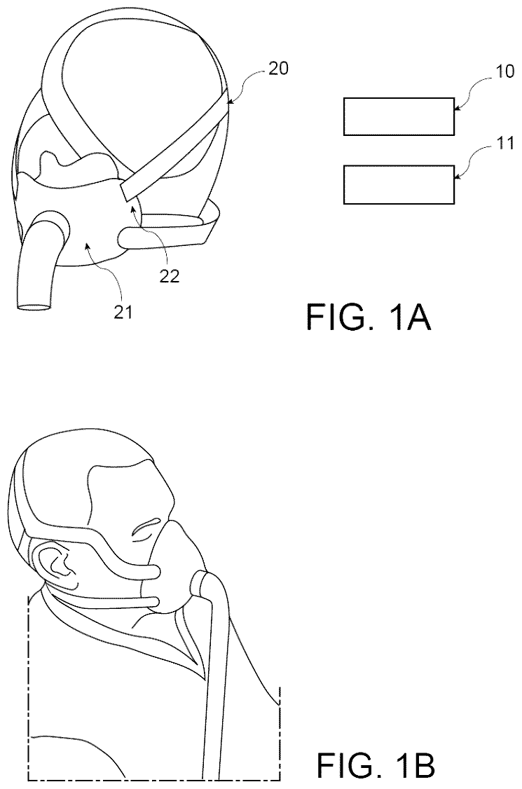

FIG. 1A shows an example of a device according to the invention. The device comprises a microphone 10, connected to a processing unit 11. The microphone is configured to record sounds produced by a user wearing a face mask 20.

FIG. 1B shows the face mask 20 worn by a user.

The processing unit 11 is programmed to process the sounds collected by microphone 10. More precisely, these are sounds produced by the user's breathing through the mask 20, during inhalation and/or exhalation.

The inventors have observed that when the mask is not correctly applied against the face, the sound resulting from the user's breathing through the mask changes. Thus, an analysis of the sound recorded by the microphone can be used to detect a possible fault in wearing the mask.

The breathing mask comprises a generally flexible and transparent shell 21, bounded by a seal 22, for example of silicone or foam. The seal is intended to be applied against the user's skin. When the mask is worn correctly, the seal forms a watertight barrier, or is considered to do so, preventing or limiting the flow of air between the internal space delimited by the shell and the user's face, and the ambient air outside the shell.

There is also an intentionally calibrated air outlet through the mask, allowing the user to exhale.

However, the mask may shift from its correct position, causing an air leakage around the seal. Such leakage is undesirable, as it compromises the ability to keep the desired pressure inside the shell, which reduces treatment efficiency.

The processing unit 11 is configured to:
process the sounds recorded by the microphone 10 to extract characteristics;
compare the extracted characteristics with a criterion, the criterion being representative a mask-wearing anomaly;
based on this comparison, determine the occurrence of a mask-wearing anomaly.

The criterion representing the mask-wearing defect is determined beforehand during a learning phase. It can be:
a spectral power threshold, in a predetermined frequency band;
a spectral power variation threshold in a predetermined frequency band;
an intensity threshold or an intensity variation threshold.

The criterion may be a periodic threshold crossing, indicating that the studied characteristic exceeds the threshold periodically, over a period compatible with a breathing period, typically a few seconds or a few tens of seconds.

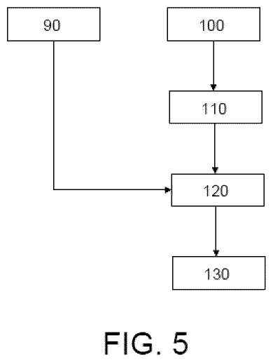

According to one possibility, the sound processing is carried out by a supervised-learning artificial intelligence algorithm, as described below in connection with FIG. 5. The criterion is therefore “implicit”, as it is taken into account in the parameterization of the algorithm. The output of the algorithm may indicate whether or not an anomaly has been detected.

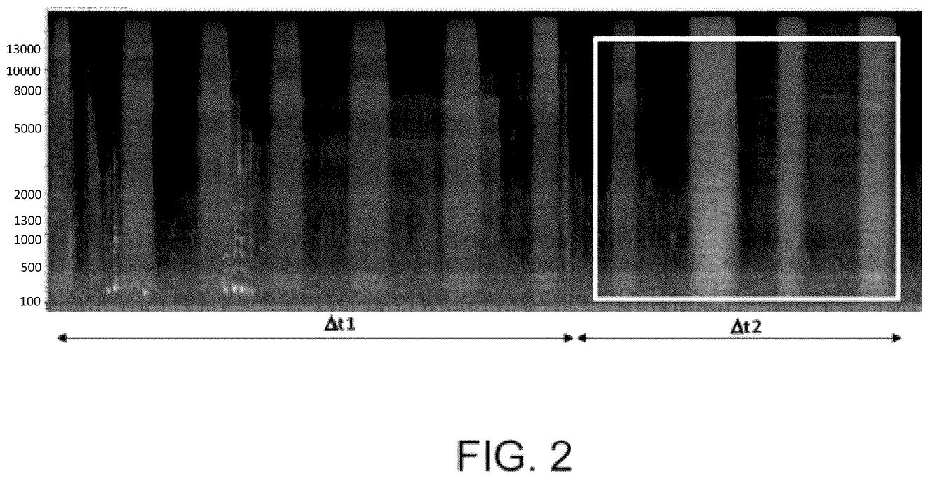

FIG. 2 shows a spectrogram of sounds recorded, in the audible domain, during a test in which a user wore a face mask correctly for a first time interval Δt₁. An anomaly was then deliberately introduced, inducing air leakage, particularly during exhalation. The anomaly was maintained for a second time interval Δt₂.

On the x-axis is time. On the y-axis is frequency (Hz). The gray level corresponds to spectral power.

In FIG. 2, the periodicity of breathing is reflected in alternating between light bands (high spectral power), corresponding to exhalations, and dark bands (low spectral power), corresponding to inspirations. The anomaly results in a periodic increase in spectral power, notably during exhalations. Spectral power is increased at frequencies above 100 Hz.

In this embodiment, a spectral power threshold criterion can be set for one or more frequencies above 100 Hz. When the measured spectral power exceeds the threshold, at any of these frequencies, an anomaly is detected. The process is more robust if different spectral bands are considered simultaneously.

In one embodiment, the processing unit is configured to detect instants during which at least one characteristic extracted from the measured sound satisfies the anomaly-related criterion. The processing unit may estimate a time period during which the characteristic satisfies the criterion. When the time period lies within a predetermined range, likely to correspond to a respiratory rhythm, the processing unit detects an anomaly. Taking the time period into account limits the occurrence of false detections. The predetermined range can be between 2 s (respiratory rate of 30 cycles per minute) and 10 s (respiratory rate of 6 cycles per minute).

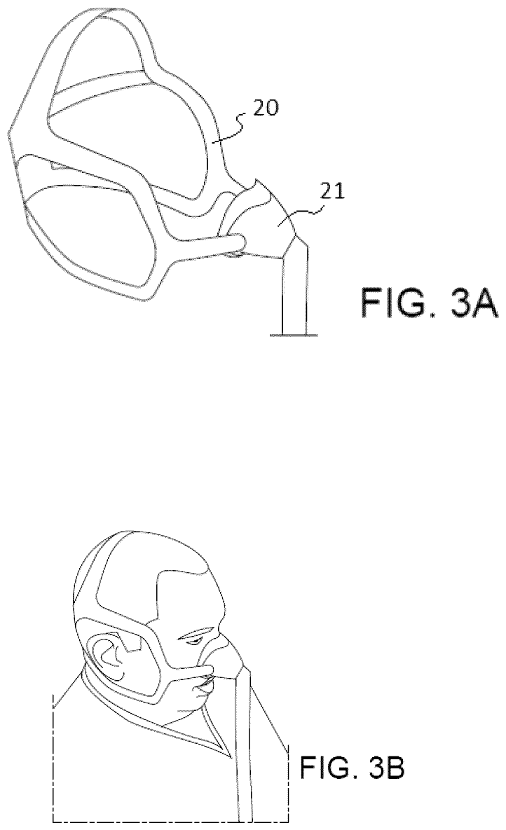

FIG. 3A shows an example of a nasal mask 20 connected to a device according to the invention. The nasal mask comprises a shell 21 covering only the nose, leaving the user's mouth uncovered.

FIG. 3B shows a nasal mask worn by a user. When using this type of mask, the mouth must remain closed, otherwise leakage will occur through the mouth. An anomaly related to the mask may occur when the mouth is open.

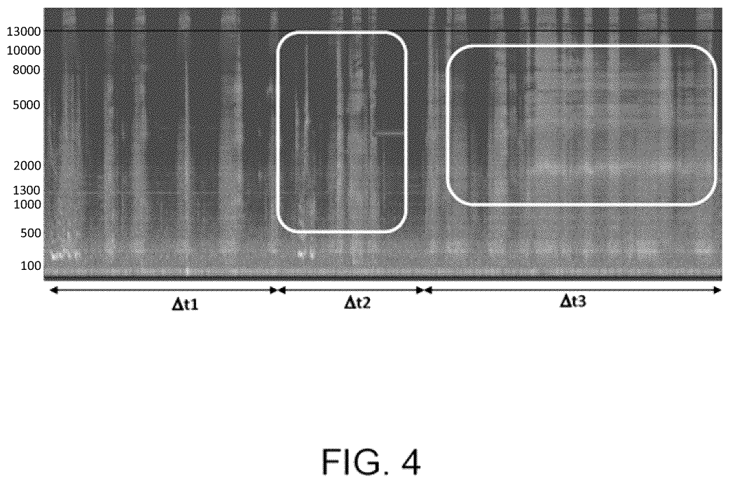

FIG. 4 shows a spectrogram of sounds recorded during a test in which a user used a nasal mask correctly for a first time interval Δt₁. A first anomaly was then deliberately introduced, during a second time interval Δt₂, corresponding to a slight opening of the mouth. A second anomaly was then deliberately introduced, during a third time interval Δt₃ corresponding to a larger mouth opening.

FIG. 4 is similar to FIG. 2: the x-axis is time, the y-axis is frequency and the gray level indicates spectral power.

As in FIG. 2, the intensity of the sound is modulated according to the respiratory rhythm, with the higher spectral power during exhalations than during inspirations. The anomalies manifest as changes in the spectrogram, and more specifically an increase in spectral power in a frequency range between 300 Hz and 10 000 Hz.

As the anomaly becomes more pronounced, there is a clear increase in spectral power between 1000 Hz and 8000 Hz. The increase in spectral power is observed in both exhalation and inspiration phases. In FIG. 4, the white box drawn during the period Δt₃ highlights a frequency band of interest.

FIG. 5 shows the main steps of sound processing implemented by the processing unit.
in a step 100, the processing unit 11 receives the sound detected by the microphone;
in a step 110, the processing unit 11 extracts one or more characteristics from the detected sound;
in a step 120, the processing unit 11 takes into account a criterion previously determined during a learning phase 90. The learning phase consists of carrying out tests for a type of mask, with and without an anomaly, in order to define which criterion or criteria correspond to an anomaly.
in a step 130, the processing unit 11 detects the occurrence of an anomaly when at least one extracted characteristic, or when each extracted characteristic, meets the anomaly-related criterion. If an anomaly is detected, an alarm signal may be generated, so as to alert the user or a team monitoring the user.

In one embodiment, the anomaly-related criterion is determined by a supervised-learning artificial intelligence algorithm, such as a neural network. The algorithm is fed either by the sound or by previously extracted sound characteristics.

The output of the algorithm is whether an anomaly is present or not. The algorithm parameters are established during the learning phase, taking into account sounds recorded respectively in the presence and absence of an anomaly. In such an embodiment, the criterion is implicitly taken into account in the algorithm. Steps 110 and 120 merge into a single step 110/120, which implements of the algorithm.

The spectrograms shown in FIGS. 2 and 4 show that a leak modifies the spectrum between 100 Hz and 13,000 Hz. Between 100 Hz and 1000 Hz, fluctuations are detected in the absence of leakage. See, for example, the fluctuations observed during the Δt1 periods in FIGS. 2 and 4. Such fluctuations may correspond to everyday noise, corresponding to activity carried out by the user, unrelated to wearing the mask. It is therefore preferable for the spectral band to extend from a wavelength greater than 1000 Hz, for example above 1100 Hz, or 1200 Hz, or 1300 Hz, or 1400 Hz, or 1500 Hz, or 2000 Hz, and below 13000 Hz. Being above 1000 Hz or 1100 Hz limits the impact of user activity on the detected sound.

The invention can be used to monitor users, both in a medical environment and at home. It does not require bulky or expensive equipment, making it particularly suited for home use.

1. A device for monitoring the wearing of a breathing mask by a user, the device comprising:
a microphone, configured to record sounds of the user exhaling or inhaling through the mask; and
a processing unit, programmed to
a) receive sounds recorded by the microphone at different times;
b) process the sound detected at each instant to extract at least one characteristic which is at least one spectral power in a frequency band;
c) according to each characteristic extracted in the processing b), detect the occurrence of a mask wearing anomaly,
wherein the frequency band starts above 1,000 Hz.

2. The device according to claim 1, wherein the processing unit is programmed so that the detecting c) comprises:
c1) taking into account at least one previously stored criterion;
c2) comparing each characteristic extracted in the processing b) with one of the criteria taken into account in c1).

3. The device according to claim 2, wherein the characteristic is a spectral power or a spectral power variation.

4. The device according to claim 3, wherein:
the characteristic is a spectral power;
the criterion is a spectral power threshold, so that the anomaly is detected when the spectral power in the frequency band exceeds the spectral power threshold.

5. The device according to claim 3, wherein:
the characteristic is a spectral power variation;
the criterion is a spectral power variation threshold, so that the anomaly is detected when the spectral power variation, in the frequency band, exceeds the spectral power variation threshold.

6. The device according to claim 2, wherein:
the criterion is a threshold;
the taking into account c1) comprises:
taking into account instants at which the characteristic crosses the threshold;
estimating a threshold-crossing time period, according to which the characteristic crosses the threshold;
in the detecting c2), the anomaly is detected when the threshold crossing time period is within a predetermined range.

7. The device according to claim 1, wherein:
the mask is a face mask, configured to cover the user's mouth and nose;
the frequency band starts above 1,100 Hz.

8. The device according to claim 1, wherein:
the mask is a nasal mask, configured to cover the user's nose without covering the mouth;
the frequency band starts above 1,100 Hz.

9. The device according to claim 1, wherein:
the processing unit implements a supervised learning artificial intelligence algorithm;
the supervised learning artificial intelligence algorithm is parameterized by a learning phase taking into account sounds detected in the absence and presence of the anomaly.

10. The device according to claim 6, wherein the predetermined range extends from 2 seconds to 10 seconds.

11. The device according to claim 1, wherein the frequency band extends up to 13,000 Hz.

12. The device according to claim 1, wherein the frequency band extends up to 8,000 Hz.

13. The device according to claim 3, wherein:
the criterion is a threshold;
the taking into account c1) comprises:
taking into account instants at which the characteristic crosses the threshold;
estimating a threshold-crossing time period, according to which the characteristic crosses the threshold;
-in the detecting c2), the anomaly is detected when the threshold crossing time period is within a predetermined range.

14. The device according to claim 4, wherein:
the criterion is a threshold;
the taking into account c1) comprises:
taking into account instants at which the characteristic crosses the threshold;
estimating a threshold-crossing time period, according to which the characteristic crosses the threshold;
in the detecting c2), the anomaly is detected when the threshold crossing time period is within a predetermined range.

15. The device according to claim 5, wherein:
the criterion is a threshold;
the taking into account c1) comprises:
taking into account instants at which the characteristic crosses the threshold;
estimating a threshold-crossing time period, according to which the characteristic crosses the threshold;
in the detecting c2), the anomaly is detected when the threshold crossing time period is within a predetermined range.

16. The device according to claim 2, wherein:
the mask is a face mask, configured to cover the user's mouth and nose, or a nasal mask, configured to cover the user's nose without covering the mouth;
the frequency band starts above 1,100 Hz.

17. The device according to claim 3, wherein:
the mask is a face mask, configured to cover the user's mouth and nose, or a nasal mask, configured to cover the user's nose without covering the mouth;
the frequency band starts above 1,100 Hz.

18. The device according to claim 4, wherein:
the mask is a face mask, configured to cover the user's mouth and nose, or a nasal mask, configured to cover the user's nose without covering the mouth;
the frequency band starts above 1,100 Hz.

19. The device according to claim 5, wherein:
the mask is a face mask, configured to cover the user's mouth and nose, or a nasal mask, configured to cover the user's nose without covering the mouth;
the frequency band starts above 1,100 Hz.

20. The device according to claim 6, wherein:
the mask is a face mask, configured to cover the user's mouth and nose, or a nasal mask, configured to cover the user's nose without covering the mouth;
the frequency band starts above 1,100 Hz.
